(19) United States
(12) Patent Application Publication
(10) Pub. No.: US 2019/0192444 A1
(43) Pub. Date: Jun. 27, 2019

(54) COMPOSITIONS AND METHODS FOR SELECTIVE GI TRACT DELIVERY

(71) Applicant: VITAL BEVERAGES GLOBAL INC., Tortola (VG)

(72) Inventors: Amir BARZILAY, Kokhav Yair (IL); Dorit ROZNER, Gedera (IL); Gilad SHACHAR, Ness Ziona (IL)

(21) Appl. No.: 16/088,168

(22) PCT Filed: Mar. 30, 2017
(86) PCT No.: PCT/IL2017/050394

§ 371 Date: Sep. 25, 2018

Related U.S. Application Data
(60) Provisional application No. 62/315,060, filed on Mar. 30, 2016.

(51) Int. Cl. A61K 9/50; A61K 45/06; A61K 47/32; A61K 47/38; A61K 47/14

(52) U.S. Cl. CPC A61K 9/5026; A61K 9/5047; A61K 9/5015; A61K 9/5089; A61K 36/488; A61K 47/32; A61K 47/38; A61K 47/14; A61K 45/06

(57) ABSTRACT

A composition is hereby provided. The present composition comprises one or more micro-sized particles in the form of a core-shell. The core comprises an active agent and ingredients subject to pH-triggered dissolution, enzymatically-degradable ingredients and/or water-insoluble ingredients. The shell comprises one or more ingredients being characterized as enteric ingredients, enzymatically-degradable, and as water-insoluble ingredients. The particles may be dispersed in a liquid or other non-dry composition.

This application claims the benefit of priority from U.S. Provisional Patent Application No. 62/315,060, filed on Mar. 30, 2016. The content of the above document is incorporated by reference in its entirety as if fully set forth herein.

### FIELD OF THE INVENTION

The present invention relates, in some embodiments thereof, to compositions and methods of using same for selective delivery of active ingredients in the gastrointestinal (GI) tract.

### BACKGROUND OF THE INVENTION

Controlled release systems for a delivery of active agents are often designed to administer drugs and other active agents to specific areas of the body. In the gastrointestinal tract, it is important that the agent will not be eliminated or become materially not bioactive during its passage in the different digestive conditions of the GI tract, and maintain sufficient bioavailability before it can exert a localized effect or to pass into the bloodstream.

Many dispersion systems are currently in use as, or being explored for use as carriers of substances, particularly biologically active compounds. These systems are designed to protect the substance from the environment during preparation for use, during oral delivery, and to provide a controlled release of the substance to a targeted area in the gastrointestinal tract.

Enteric coated formulations (e.g. formulations with non or minimal release of an active ingredient prior to encountering the digestive conditions of the distal small intestines) have been widely used for many years to protect drugs or other biologically active agents administered orally, as well as to delay release. Several microsphere formulations have been proposed as a means for oral drug delivery. These enteric coated formulations are primarily in the form of dry formulations (e.g. delivered in the form of tablets, capsules, etc.).

Despite the great progress and advancements in the area of various drug delivery systems, many drugs and dietary supplements are prone to poor bioavailability when delivered orally, due to poor water solubility and biological barriers, for example, at the enterocyte level.

International Patent Application Publication No. WO 2006/039022 discloses a composite formulation being developed for selective, high efficacy delivery to specific regions of the mouth and gastrointestinal tract. The formulation in WO 2006/039022 is typically in the form of a tablet or capsule (e.g. maintained in a dry form until consumption), which may include microparticles or beads, where the drug is absorbed in enhanced amounts in the small intestines relative to a formulation absent of the bioadhesive and/or controlled release elements.

There still remains a need for active agent (e.g., drug, active or dietary supplement) delivery systems that can survive the harsh conditions in the GI tract, and yet effectively deliver the active agent.

### SUMMARY OF THE INVENTION

The present invention relates, inter alia, to a composition which can be used to selectively deliver active agent(s) (e.g., pharmaceutical and natural APIs and other active ingredients) in the gastro-intestinal (GI) tract. In some embodiments, a composition as described herein targets a specific location and/or region within the GI tract.

According to an aspect of some embodiments, there is provided a composition comprising at least one microparticle, wherein the microparticle is in the form of a core-shell, and wherein:
the core comprises:
(a) at least one active agent; and
(b) at least one pH-triggered ingredient, at least one enteric ingredient, or at least one enzymatically-degradable ingredient, or any combination thereof; and
the shell comprises one or more water-insoluble and enzymatically-degradable ingredients, wherein the weight ratio of the active agent and the sum of (b) ranges from 1:1,000 to 10:1.

In some embodiments, the shell further comprises (i) at least one pH-triggered ingredient or (ii) at least one enteric ingredient.

In some embodiments, the active agent is water-soluble.

In some embodiments, the active agent is water-insoluble.

In some embodiments, the active agent is selected from the group consisting of: pharmaceutical APIs, active ingredients, nutraceuticals, food supplements, food additives, herbals, plant extracts, medicaments, homeopathic agents, and any combination thereof.

In some embodiments, the shell comprises two or more layers, wherein each layer comprises a different composition. In some embodiments, the shell further comprises one or more active agents.

In some embodiments, the composition comprises pH-triggered ingredient or an enteric ingredient, and the enzymatically-degradable ingredient.

In some embodiments, a weight ratio of the at least one active agent and the shell ranges from 100:1 to 1:100.

In some embodiments, the composition comprises a plurality of microparticles.

In some embodiments, each microparticle is characterized by different core and shell properties.

In some embodiments, the composition further comprises a liquid.

In some embodiments, the liquid is selected from the group consisting of: water, alcohol, and any combination thereof.

In some embodiments, the alcohol is selected from ethanol and methanol or a mixture thereof.

In some embodiments, the active agents are encapsulated and selected from the group consisting of Sugar, Fructose, Glucose, Carbohydrates, Betaine, Choline, Bi vitamins, Vitamin C, Vitamin D, Vitamin E, Coenzyme A, Cysteine/N-Acetyl-L-cysteine, Carnitine, Kudzu (Puerarin), Huperzia (Huperzine), Guarana (Caffeine, Theophylline), Alpha lipoic acid, Curcuma Longa (Curcumin), Piper Longum (Piperine), Astaxanthin, Quercetin, Resveratrol, Ginkgo Biloba (Bilobalide and Ginkgolides), Ginseng (Ginsenosides), Bacopa Monnieri (Bacosides), Ginger (Gingerols, Shogalos), Butterbur (Petasins), Feverfew (Parthenolides), White Willow Bark (Salicin/Salicylic acid), Cannabidiol, and Grape seed (Proanthocyanidins) or any combination thereof.

In some embodiments, the composition comprises one or more non-encapsulated active agents.

In some embodiments, the non-encapsulated active agents are selected from Sugar, Fructose, Glucose, Carbohydrates, Betaine, Choline, Bi vitamins, Vitamin C, Vitamin D, Vitamin E, Coenzyme A, Cysteine/N-Acetyl-L-cysteine, Carnitine, Kudzu (Puerarin), Huperzia (Huperzine), Guarana (Caffeine, Theophylline), Alpha lipoic acid, Curcuma Longa (Curcumin), Piper Longum (Piperin), Astaxanthin, Quercetin, Resveratrol, Ginkgo Biloba (Bilobalide and Ginkgolides), Ginseng (Ginsenosides), Bacopa Monnieri (Bacosides), Ginger (Gingerols, Shogalos), Butterbur (Petasins), Feverfew (Parthenolides), White Willow Bark (Salicin/Salicylic acid), Cannabidiol, and Grape seed (Proanthocyanidins) or any combination thereof.

In some embodiments, the liquid further comprises a surfactant.

In some embodiments, the microparticles are dispersed in the liquid.

In some embodiments, the liquid further comprises one or more non-encapsulated supplementary ingredients such as flavoring agents.

In some embodiments, the composition is characterized by pH below 7. In some embodiments, the pH is below 4.

In some embodiments, the enteric ingredients comprise a water soluble or water insoluble polymeric material selected from the group consisting of: hydroxypropylmethyl cellulose phthalate, hydroxypropylmethyl cellulose acetate succinate, methacrylic acid-methyl methacrylate copolymer, ethyl methacrylate-methyl methacrylate-chloro-trimethylammonium ethyl methacrylate copolymer, cellulose acetate phthalate, cellulose propionate phthalate, cellulose acetate maleate, polyvinyl acetate phthalate, polyvinyl alcohol phthalate, styrene-acrylic acid copolymer, methyl acrylate-methacrylic acid copolymer, or a water-soluble ingredient consisting of acetyl cellulose, methyl cellulose, hydroxypropyl cellulose, hydroxypropylmethyl cellulose, carboxymethyl cellulose, hydroxypropyl methylcellulose (HPMC), HPMPC, Carbomers, PEGs, Prolamin proteins (Zein, Gluten, Kafirin, etc.), Shellacs, fats (e.g., Coconut oil, Palm oil, Carnauba wax, Stearic acid, Sunflower oil), Gelatin, Soy proteins, Pea proteins (Globulin), Vegetable proteins, Starches, Dextran, Maltodextrin, Cyclodextrin, Whey, Casein, Guar gum, gum Arabic, Pectin, Amylose, Chitosans, Alginates, Hydrogels, HMPC, HPMPC, PVA, Polyethylene glycol (PEG), Carbomers, Polymethacrylate, Ethyl Cellulose, Methyl Cellulose and any combination thereof.

In some embodiments, the shell comprises one or more materials selected from: HPMC, HPMPC, Carbomers, PEGs, Prolamin proteins (Zein, Gluten, Kafirin, etc.), Shellacs, fats (Coconut oil, Palm oil, Carnauba wax, Stearic acid, Sunflower oil), Gelatin, Soy proteins, Pea proteins (Globulin), Vegetable proteins, Starches, Dextran, Maltodextrin, Cyclodextrin, Whey, Casein, Guar gum, gum Arabic, Pectin, Amylose, Chitosans, Alginates, Hydrogels, HMPC, HPMPC, PVA, PEGs, Carbomers, Polymethacrylate, Ethyl Cellulose and Methyl Cellulose.

In some embodiments, a dimension of the microparticle is characterized by a diameter of 1 to 300 microns. In some embodiments, the dimension of the microparticle is characterized by a diameter of 10 to 100 microns. In some embodiments, the dimension of the microparticle is characterized by a diameter of 25 to 75 microns.

In some embodiments, the composition is formulated for at least one dissolution trigger and/or one selective release profile in a GI tract.

In some embodiments, the selective release is within a time limit, temperature threshold, pH threshold, ionic strength, or enzymatic activity, or any combination thereof.

In some embodiments, the composition is in the form of: powder, a beverage, shake, foam, capsule, a tablet, a bar or a gel.

In some embodiments, the composition is characterized by a sigmoidal pattern of controlled release of at least one active agent within a gastrointestinal (GI) tract.

In some embodiments, at least two microparticles from a plurality of the microparticles are characterized by having a different dissolution trigger and/or a different sigmoidal pattern of controlled release of the active agent in the GI tract.

According to an aspect of some embodiments, there is provided a kit for oral administration, comprising:

(a) the disclosed composition, and

(b) instructions for use.

According to an aspect of some embodiments, there is provided a use of the disclosed composition for providing an effective dose of an active agent to one or more target sites within the gastrointestinal (GI) tract.

In some embodiments, the disclosed composition is for oral administration.

According to an aspect of some embodiments, there is provided a method for treating an acute, transient or chronic disorder, comprising administrating the disclosed composition to an individual.

In some embodiments, the disclosed composition is specifically formulated for controlled release of the at least one active agent in a specific region of the gastrointestinal (GI) tract of a subject.

According to an aspect of some embodiments, there is provided a process for preparing the disclosed microparticle. In some embodiments, the process comprises:

(A) blending the active agent and at least one from (i) and (ii) in a solution comprising organic solvent and/or water, thereby forming a core solution;

(B) dissolving the one or more enzymatically-degradable water-insoluble; and

(C) concurrently spray-drying the solutions, each in a separate channel, thereby forming the microparticle.

In some embodiments, the one or more enzymatically-degradable and/or water-insoluble ingredients are in amount that ranges from 1% to 99%, by total weight of the core solution.

In some embodiments, the organic solvent is selected from the group consisting of pharma-grade solvents and food-grade solvents, including, but not limited to: ethanol, methanol, acetone, and any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C illustrates typical dissolution graphs of gastric target release showing the release profile of Vitamin B5 (FIG. 1A), Calcium Pyruvate (FIG. 1B), and of Kudzu (FIG. 1C).

FIG. 2 is a graph showing typical enteric release profiles of the active agent (Kudzu).

FIG. 3 is a graph showing the release profile of the active agent (Kudzu) when Zein protein, an encapsulation ingredient subject to enzymatic triggering and disintegration, was used to prevent the release of the active agent disclosed in the microparticle when reconstructed into an acidic liquid formula which is absent of enzymatic triggering conditions.

FIG. 4 is a graph showing the release profile of the active agent (Kudzu) when the microparticles have the core (Kudzu/Shellac 1%+Zein 4%) with no shell protection.

FIG. 5 is a graph showing the release profile of the active agent (Kudzu) when the microparticles have a core component with the enteric ingredient (Kudzu/Shellac 1%+Ethocel 10%) without incorporating an enzymatic-degradable ingredient.

FIG. 6 is a graph showing the release profile of the active agent (Kudzu) when the formulation was produced in an open-loop system spray dryer with a reverse coating approach.

In the FIGS bars represent standard deviation of three independent experiments.

### DETAILED DESCRIPTION

The present invention relates, inter alia, to a composition which can be used to selectively deliver active agent(s) (e.g., APIs and other active ingredients) in the gastro-intestinal (GI) tract. In some embodiments, a composition as described herein is selectively targeted to or targets a specific location and/or region within the GI tract. In some embodiments, a composition as described herein selectively targets multiple or specific locations, regions and/or triggers within the GI tract, as described hereinbelow. Non-limiting exemplary locations and/or regions within the GI tract are selected from mouth, stomach, duodenum, jejunum, ileum, cecum, colon and any combination thereof.

It is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting in any manner.

As further described hereinbelow, the disclosed composition may be manufactured via spray drying with a three fluid or four fluid or five fluid injectors, or via a combination of spray drying followed by fluidized bed, or via a fluidized bed. Injectors with multiple fluid and gas channels may incorporate more than a single fluid nozzle and are capable of generating, in a single step, a core component containing the active ingredient or a matrix containing the active ingredient surrounded by at least one layer of a coating shell made from, for example and without limitation, cellulose derivatives, proteins, methacrylates, phthalates and any other coating materials known in the food and/or pharmaceutical industry.

The final microencapsulated product may be in the form of a powder that is dispersed into a non-dry delivery composition/environment, e.g., an acidic liquid beverage, shake, a gel or any other forms of non-dry vehicles known in the art.

Since the microencapsulated powder may be water-insoluble and may include a high dose of active ingredients where at least one of the active ingredients is poorly water-soluble, parameters that optimize particles dispersion, active agent loading within the particle, and minimize of a negative mouth sensation (organoleptic) of the microencapsulated powder are selected.

Suspended or dispersed water-insoluble particles in liquid with a particle diameter above 100 microns typically produce an unpleasant sand-like gritty sensation in the mouth, thus generating an unfavorable consumption experience.

The present inventors have contemplated encapsulating active ingredients in microcapsules with different coatings which isolate (in contrast to e.g., mask) the odors and the flavors of active ingredients with unpleasant tastes and odors. Such microcapsules also enable loading of an effective amount of active ingredients when dispersed in a liquid unit of e.g., from several milliliters to several hundreds of milliliters, in a drink, beverage, shake, a gel or in other non-dry delivery forms e.g., as described herein.

As is further described below, microencapsulation at the selected particle size/diameter may also be selected so as to minimize or negate the gritty oral sensation often accompanied with consumption of suspended or dispersed powders in no-dry delivery systems. Such gritty oral sensation of a dispersed powder in a drink, beverage, a shake or a gel is a major obstacle for obtaining a satisfactory positive consumption experience by the user.

In addition, the present invention's microencapsulation approaches were designed for the purpose of selectively releasing different active ingredients (e.g., APIs, other actives and/or dietary supplements) throughout the GI tract (as described in the Examples section which follows).

The principles and operation of the present invention may be better understood with reference to the drawings and accompanying descriptions and examples.

The Composition:

According to one aspect of the present invention there is provided a composition comprising at least one microparticle, wherein the at least one microparticle is in the form of a heterostructure. In some embodiments, the heterostructure is in the form of a matrix, core-shell or core-shell structure (also referred to as “encapsulated particles”).

In some embodiments, the microparticle can be generally shaped as a sphere, a rod, a cylinder, a ribbon, a sponge, and any other shape, or can be in the form of a cluster or an aggregate of any of these shapes, or can comprise a mixture of one or more shapes.

In some embodiments, the core comprises at least one active agent and at least one from (i), (ii) and (iii):

(i) one or more enteric ingredients, and

(ii) one or more enzymatically-degradable ingredients;

(iii) one or more pH-triggered ingredient.

In some embodiments, the composition comprises (i).

In some embodiments, the composition comprises (ii).

In some embodiments, the composition comprises (iii).

In some embodiments, the composition comprises (i) and (ii). In some embodiments, the composition comprises (ii) and (iii). In some embodiments, the composition comprises (i) and (iii). In some embodiments, the composition comprises (i), (ii), and (iii). In some embodiments, the composition comprises two of (i) to (iii).

In some embodiments, the shell comprises one or more ingredients being characterized as enzymatically-degradable, enteric and/or as water-insoluble ingredients.

In some embodiments, the shell further comprises one or more active agents described hereinthroughout.

In some embodiments, the shell comprises one or more ingredients being enteric ingredients.

In some embodiments, the enteric ingredients are also enzymatically-degradable ingredients.

In some embodiments, the enteric ingredients are not enzymatically-degradable ingredients.

The term “core-shell structure” generally refers to a solid material, wherein the solid material is a particulate material, and wherein individual particle(s) is characterized by containing at least two different types of materials which may be distinguished from one another by their shape, by their diameter, by their composition, by their structure and/or by their placement within the particle, wherein one or more materials of a certain type are contained in the interior portion of the particles. The interior portion is designated by the term “core” or “matrix” or “inner matrix”. One or more materials of a certain type which may be distinguished from the one or more materials contained in the interior portion are contained in the outer portion of the particles. The outer portion comprising the surface is designated by the terms “shell” or “coating layer” or “encapsulation layer”.

In some embodiments, the core-shell structure is a closed structure.

The term “closed” as used herein, is a relative term with respect to the size, the shape and the particle or composition of two entities, namely an entity that defines an enclosure (the enclosing entity) and the entity that is being at least partially enclosed therein. In general, the term “closed” refers to a morphological state of an object which has a discrete inner (e.g., the core) and outer surfaces which are substantially disconnected, wherein the inner surface constitutes the boundary of the enclosed area. The enclosed area may be at least partially secluded from the exterior area of space.

In some embodiments, the shell and/or the core is designed to prevent the release of foul-tasting and/or water insoluble and/or water soluble ingredients (e.g., active agents) into the non-dry delivery composition/environment (e.g., a liquid, drink, beverage, shake, gel, or other non-dry delivery system) for a sufficient time period for a common individual or consumer to completely consume e.g., a 1-10 ml, 10-100 ml, or 300-500 ml or 500-1,000 ml serving. Herein, by “sufficient time period”, it is meant to refer to at least e.g., 1 min, 5 min, 10 min, 15 min, 20 min, 30 min, 40 min, 50 min 60 min, 90 min, 120 min, 180 min, 240 min, 300 min, 360 min, 420 min, 480 min or 600 min, 720 min, 1 day, 7 days, 14 days, 30 days, 60 days, 90 days, 180 days or 365 days, including any value therebetween.

Herein, the term “liquid” may refer to any liquid suitable for human consumption, including, without being limited thereto, water, juice, milk, tea, and alcohol.

Hereinthroughout, the term “microparticle” describes a particle featuring a size of at least one dimension thereof (e.g., diameter, length) that ranges from about 1 micrometer to 1,000 micrometers (microns). In some embodiments, the particle's size ranges from 1 to 300 microns. In some embodiments, the particle's size ranges from 10 to 200 microns. In some embodiments, the particle's size ranges from 30 to 100 microns, e.g., 30 μm, 40 μm, 50 μm, 60 μm, 70 μm, 80 μm, 90 μm, 100 μm, including any value and range therebetween.

According to another aspect of some embodiments, there is provided a composition comprising at least one microparticle, wherein the microparticle is in the form of a matrix, as demonstrated e.g., in FIG. 4, and wherein:
the matrix comprises:
(a) at least one active agent; and
(b) at least one pH-triggered ingredient, at least one enteric ingredient, or at least one enzymatically-degradable ingredient, one or more water-insoluble and enzymatically-degradable ingredients or any combination thereof; and wherein the weight ratio of the active agent and the sum of (b) ranges from 1:1,000 to 10:1.

In some embodiments, the active agent in the matrix is water-soluble.

In some embodiments, the active agent in the matrix is water-insoluble.

In some embodiments, the active agent in the matrix is selected from pharmaceutical APIs, active ingredients, nutraceuticals, food supplements, food additives, herbals, plant extracts, medicaments, homeopathic agents, and any combination thereof.

In some embodiments, the composition comprises pH-triggered ingredient or an enteric ingredient, and the enzymatically-degradable ingredient.

As described hereinabove, in some embodiments, the advantages of the disclosed technological approach is that in given optimal parameters (e.g., very small particles) the size/diameter of the particles are below the sensory threshold in an oral cavity. By “sensory threshold” it is meant to refer to the weakest stimulus that an organism can detect. That is, if the stimulus falls below the threshold, the subject does not experience it.

In some embodiments, an existence of a plurality of the disclosed microsized particles dispersed in a non-dry delivery system/environment (e.g., a liquid, a drink, a beverage, a shake, a gel or any other non-dry delivery system) is below the sensory threshold of the tongue (dictated by e.g., bumps on the tongue called fungiform papillae) and all other locations in the oral cavity and the pharynx capable of sensing a particle, and is therefore not sensed by a subject as particles.

In some embodiments, the bulk density (gr/ml) of the microparticles is from 0.1 to 0.9, including any value therebetween. In some embodiments, the bulk density of the microparticles is from 0.2 to 0.8. In some embodiments, the bulk density of the microparticles is from 0.3 to 0.7, including any density value therebetween. In some embodiments, the bulk density of the microparticles is from 0.4 to 0.6 including any density value therebetween.

In some embodiments, the size of the particle described herein represents an average or a median size of a plurality of microparticles.

In some embodiments, the average or median size (e.g., diameter, length) ranges from about 1 micrometer to 500 micrometers. In some embodiments, the average or median size ranges from about 1 micrometer to about 300 micrometers. In some embodiments, the average or median size ranges from about 1 micrometer to about 200 micrometers. In some embodiments, the average or median size ranges from about 1 micrometer to about 100 micrometers.

In some embodiments, the average or median size is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 40 μm, about 42 μm, about 44 μm, about 46 μm, about 48 μm, 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 400 μm, about 420 μm, about 440 μm, about 460 μm, about 480 μm, or about 500 μm, including any value and size range therebetween.

In some embodiments, the matrix or shell comprises one or more ingredients which are insoluble in liquid without the presence of enzymes and/or specific pH conditions.

In some embodiments, the matrix or shell comprises one or a combination of ingredients being characterized as e.g., enzymatically-degradable and/or pH-degradable.

In some embodiments, the enzymatically-degradable ingredients are selected from the group consisting of polyethylene glycol, enzymatically degradable peptides and enzymatically degradable proteins, and, synthetic polymers.

In some embodiments, the polymers are selected from poly(ethylene oxide) (PEO), poly (ethylene glycol) (PEG) and copolymers with poly(propylene oxide) (PEG-co-PPG), poly(vinyl alcohol) (PVA), poly(vinylpyrrolidone) (PVP), poly(ethyloxazoline) (PEOX), polyaminoacids, and pseudopolyamino acids, and copolymers of these polymers.

In some embodiments, the one or more ingredients are not soluble in water (also referred to as: “water-insoluble”). By “not soluble in water” or “water-insoluble” it is meant that the Kₛₚ of the ingredient (or agent) in water is below 10⁻¹⁰, or in another embodiment below 10⁻⁹, or in another embodiment below 10⁻⁸, at least 10⁻⁷, or in another embodiment below 10⁻⁶, or in another embodiment below 10−5, or in another embodiment below 10⁻⁴, or in another embodiment below 10⁻³, or in another embodiment below 10⁻², or in another embodiment below 10⁻¹.

In some embodiments of the present invention, each microparticle can include more than one coating/encapsulation layer.

In some embodiments, the shell comprises two or more layers, (e.g., 2, 3, 4, or 5 layers). In some embodiments, each layer comprises a different ingredient or at least one different ingredient. In some embodiments, the term “different” refers to different dissolvability profile of each ingredient so as to allow e.g., obtaining stepwise release profiles of the active agent(s).

In some embodiments, the term “enzymatically-degradable” is intended to mean that the stated ingredient is capable of being cleaved or digested, either partially or extensively, by enzymes present in different sections of the GI tract. For example, and without limitation, polyamino-acids, lipids, fats, proteins, polysaccharides, carbohydrates and nucleotides are usually enzymatically degradable.

In some embodiments, an enzymatically-degradable ingredient in the core, matrix or in the shell is degradable by only a specific enzyme present in a specific GI location (e.g., mouth, stomach, duodenum, jejunum, ilium, secum or the colon). In some embodiments, an enzymatically-degradable ingredient in the core, matrix and/or in the shell is degradable by a specific enzyme present in more than one specific GI location. In some embodiments, an enzymatically-degradable ingredient in the core, matrix and/or the shell is degradable by a more than one enzyme presents in more than one specific GI location.

In some embodiments, the core, matrix and/or the shell incorporate at least one ingredient that is both water-insoluble and enzymatically-degradable.

In some embodiments, a weight ratio of the active agents and a total of (i), (ii) and (iii) as defined hereinabove is within the range of from 1:1,000 to 50:1, for example, 1:1,000, 1:900, 1:800, 1:700, 1:600, 1:500, 1:400, 1:300, 1:200, 1:100, 1:50, 1:40, 1:30, 1:20, 1:10, 1:1, 2:1, 3:1, 4:1 5:1, 10:1, 20:1 30:1, 40:1 or 50:1, including any value or range therebetween.

In some embodiments, the core, matrix and/or the shell further comprise one or more enteric ingredients. In some embodiments, the shell comprises one or more enteric ingredients, which are absent in the core.

In some embodiments, the term “enteric ingredient” refers to a barrier (e.g., a polymeric barrier) applied to any orally-delivered medication or other active or dietary supplement, that prevents its dissolution or disintegration in the oral and/or in one or more proximal sites/regions in the gastric environment (e.g. the mouth, the esophagus, the stomach and the proximal part of the small intestines).

In some embodiments, the enteric material is a polymer, e.g., cellulose or a derivative thereof (e.g., ethyl cellulose, hydroxypropylcellulose (HPC)).

In some embodiments, the enteric ingredient is characterized by pH (between pH 4.5-pH 7.6) dependent solubility. In some embodiments, the enteric ingredient is characterized by non-pH dependent solubility. In some embodiments, the enteric ingredient is not susceptible to enzymatic degradation. In some embodiments, the enteric ingredient is characterized by water/liquid swellability.

In some embodiments, the term: “enteric ingredient” further encompasses a reversed enteric material, e.g., a material characterized by increased solubility at low (e.g., acidic) pH than at higher pH. Non-limiting example of reversed enteric material is Zein protein.

Non-limiting examples of enteric ingredients or materials are one or more polymeric materials selected from the group consisting of: hydroxypropylmethyl cellulose phthalate, hydroxypropylmethyl cellulose acetate succinate, methacrylic acid-methyl methacrylate copolymer, ethyl methacrylate-methyl methacrylate-chloro-trimethylammonium ethyl methacrylate copolymer, cellulose acetate phthalate, cellulose propionate phthalate, cellulose acetate maleate, polyvinyl acetate phthalate, polyvinyl alcohol phthalate, styrene-acrylic acid copolymer, methyl acrylate-methacrylic acid copolymer, or a water-soluble ingredient consisting of cellulose or a derivative thereof, acetyl cellulose, methyl cellulose, hydroxypropyl cellulose, hydroxypropylmethyl cellulose, carboxymethyl cellulose, (Hydroxypropyl)methyl cellulose (HPMC), Carbomers, Prolamin proteins (e.g., Zein, Gluten, Kafirin,), Shellacs, fats (e.g., Coconut oil, Palm oil, Carnauba wax, Stearic acid, Sunflower oil), Gelatin, Soy proteins, Pea proteins (Globulin), Vegetable proteins, Starches, Dextran, Maltodextrin, Cyclodextrin, Whey, Casein, Guar gum, gum Arabic, Pectin, Amylose, Chitosans, Alginates, Hydrogels, HMPC, HPMPC, PVA, Polyethylene glycol (PEG), Carbomers, Polymethacrylate, Ethyl Cellulose, Methyl Cellulose, and any combination thereof.

In some embodiments, an active agent is administered, for example, to treat a temporary, transient or acute disease state or physiological condition, such as a disorder, an imbalance or a nutritional deficiency.

In some embodiments, the term “active agent” is understood to include chemical, biological, dietary, nutritional or pharmaceutical entities including any natural or synthetic chemical or biological substance. Typical active entities include, but are not limited to, active pharmaceutical ingredients, antibodies, antigens, biological materials, chemical materials, chromatogenic compounds, contrasting agents, drugs, enzymes, fluorescent probes, immunogenes, indicators, ligands, nucleic acids, nutrients, peptides, physiological media, proteins, fats, lipids, oils, carbohydrates, amino acids, selective toxins, and toxins.

In some embodiments, the active agent is selected from, but is not limited to, the group consisting of nutraceuticals, food supplements, food additives, plant extracts, medicaments, herbals, homeopathic agents, and any combination thereof.

In some embodiments, the active agent is a dietary supplement or a specific active molecule within a dietary supplement. In some embodiments, dietary supplement refers to a product taken orally that contains an ingredient that is intended to supplement an individual's diet and is not considered food (i.e. are not typically an energy source). A dietary supplement may be a vitamin, a mineral, herb or other botanical or extract thereof, one or more amino acids, one or more proteins, one or more lipids, or any other non-food substance capable of supplementing a diet.

Exemplary embodiments of the active agent are discussed hereinbelow in the Examples Section.

In some embodiments, the active agent is encapsulated as discussed hereinabove (e.g., is a part of the core of the disclosed microparticle).

Non-limiting exemplary encapsulated active agents are selected from, without being limited thereto, Cysteine/N-Acetyl-L-cysteine, Kudzu (Puerarin), Huperzia (Huperzine), Guarana (Caffeine, Theophylline), Alpha lipoic acid, Curcuma Longa (Curcumin), Piper Longum (Piperin), Quercetin, Resveratrol and Ginseng (Ginsenosides), Bacopa Monnieri (Bacosides), Ginger (Gingerols, Shogalos), White Willow Bark (Salicin/Salicylic acid), Cannabidiol and Grape seed (Proanthocyanidins) or any combination thereof.

In some embodiments, the composition further comprises one or more active agents which are not encapsulated.

Non-limiting exemplary non-encapsulated active agents are dietary supplements, such as Vitamin B, Vitamin C, Magnesium salt, Zinc salt, Carnitine/Acetyl-L-carnitine, Pyruvate salt, Cysteine, N-Acetyl-L-cysteine, Kudzu (Puerarin), Huperzia (Huperzine) and Guarana (Caffeine, Theophylline), or any combination thereof.

Non-limiting exemplary dietary supplements which, in some embodiments, may be encapsulated, include, but are not limited to, Alpha Lipoic Acid, Folic acid, Vitamin B1, Vitamin B2, Vitamin B3, Vitamin B5, Vitamin B6, Vitamin B7, Vitamin B9, Vitamin B12, Vitamin D, Vitamin E, Glutathione, Carnitine/Acetyl-L-Carnitine, Cysteine/N-Acetyl-L-Cysteine, Copper salt, Selenium salt, Magnesium salt, Zinc salt, Pyruvate salt, Co-enzyme A, Curcumin, Piperin, Quercetin, Resveratrol, Sesamin, Sulforaphane, Astaxanthin, Lemon (Limonene), Grape seed (Proanthocyanidins), Huperzia (Huperzine), Hovenia Dulcis (Dihydromyricetin), Pueraria Lobata (Puerarin), Ginseng (Ginsenosides), Ginkgo Biloba (Bilobalide, Amentoflavone, Ginkgolides), Guarana (Caffeine, Theophylline), Bacopa Monnieri (Bacosides), Ginger (Gingerols, Shogalos), Feverfew (Parthenolides), Butterbur (Petasins), White Willow Bark (Salicin/Salicylic acid), Mango (Mangiferin), Betaine (TMG), SAMe, Choline salt, Creatine, Cannabidiol, Fructose and Glucose.

Further specific non-limiting exemplary dietary supplements which, in some embodiments, are partially encapsulated and partially not encapsulated include, but are not limited to, Fructose, Glucose, Betaine (TMG), Choline salt, Lemon powder, Citric acid, Sucralose, Caramel, Thickener (e.g., 7HOF), Gum Arabic, Silicon dioxide, Betaine (TMG), SAMe, Choline salt, Creatine, Fructose, Glucose, Vitamin D, Vitamin E, Glutathione, Copper salt, Selenium salt, Co-enzyme A, Sesamin, Sulforaphane, Astaxanthin, Lemon (Limonene), Ginkgo Biloba (Bilobalide, Bacopa Monnieri (Bacosides), Amentoflavone, Ginkgolides), Feverfew (Parthenolides) and Mango (Mangiferin).

In some embodiments, by “vitamin B” it is meant to refer to vitamin B 1, vitamin B2, vitamin B3, vitamin B5, vitamin B6, vitamin B7, vitamin B9, vitamin B12, or any combination thereof. In some embodiments, by “vitamin B” it is meant to refer to vitamin B complex.

It is to be understood that one or more active agents referred to as “non-encapsulated active agents”, as listed above, may be encapsulated, in some embodiments.

In some embodiments, the active agent is water-soluble. As used herein, the term “water-soluble” describes a substance which is capable of forming a homogeneous mixture in water. The homogeneous mixture may be a solution, in which the substance dissolves in water.

In some embodiments the active agent is highly soluble in water.

Without being bound by any particular mechanism, when the active agent is highly soluble in water (for example, vitamin B6), it “leaks” into the non-dry (e.g., liquid) medium (e.g., beverage, shake, gel, etc.). To ensure the desired release profile and the bioavailability of the active agent in the GI tract, a shell incorporating more than one encapsulation agent/layer may be desired.

In some embodiments the active agent is partially soluble in water.

In some embodiments the active agent is non-soluble in water.

In some embodiments, the term “water-soluble” refers to a substance which is materially not water-insoluble, as defined herein above.

For example, water-solubility is characterized by at least 5 weight percent, at least 10 weight percent, at least 20 weight percent or at least 25 weight percent.

In some embodiments, the term “water-solubility” refers to the maximal concentration of a substance in water, at which the substance is water-soluble.

In some embodiments, the at least one active agent and the shell are in a weight ratio that ranges from 1:20 to 20:1. In some embodiments, the weight ratio is 1:20, 1:15, 1:10, 1:5, 1:3, 1:2, 1:1, including any value and range therebetween.

In some embodiments, the composition comprises a plurality of the disclosed microparticle (also referred to as “microparticles”, for simplicity).

In some embodiments, at least some, and in some embodiments, most of the microparticles are generally shaped as spheres.

In some embodiments, and at least 10%, 20%, 30%, 40%, 50%, 60%, 70%, 80%, 90%, 95%, 98%, 99%, 99.9%, or all of the microparticles have an average size that varies within ±20%.

In some embodiments, at least 10%, 20%, 30%, 40%, 50%, 60%, 70%, 80% , 90%, 95%, 98%, 99%, 99.9%, or all of the microparticles have an average size that varies within ±30%.

In some embodiments, at least 10%, 20%, 30%, 40%, 50%, 60%, 70%, 80% , 90%, 95%, 98%, 99%, 99.9%, or all of the microparticles have an average size that varies within ±40%.

In some embodiments, and at least 10%, 20%, 30%, 40%, 50%, 60%, 70%, 80%, 90%, 95%, 98%, 99%, 99.9%, or all of the microparticles have an average size that varies within ±50%.

As is mentioned hereinabove, the present composition can include more than one type of a microparticle in e.g., a single powdered product. Different types of microparticles can include different matrices (e.g., different active ingredient in a core without a shell/coating), different cores (i.e. different active ingredient in a matrix with at least one additional ingredient) and/or a different type of shells/coatings (facilitating selective release of more than one active ingredient).

In some embodiments, the disclosed microparticle may comprise different active agents each being characterized by a different solubility profile in water, fluid, digestive fluid or any other liquid.

In some embodiments, the active agent desired to be delivered to the lower gastrointestinal tract region has a selective trigger and/or release profile in the lower gastrointestinal tract. In some embodiments, the active agent is desired to be delivered to upper gastrointestinal tract has a selective trigger and/or release profile in the upper gastrointestinal tract.

In some embodiments, the selective release is triggered by an environmental trigger, as described hereinbelow under Bioactivity of the Composition.

For example, and without being limited thereto, a single powdered unit dose may include:

(a) a microparticle facilitating rapid release of active agent X starting in the stomach;

(b) a microparticle facilitating delayed release of active agent Y starting in the Duodenum (sustained release between 15-120 minutes post consumption); and

(c) a microparticle facilitating delayed release of active agent Z starting in the colon along (sustained release between 60-480 minutes post consumption).

Each microparticle may have minimal release of the active agent in a liquid.

Each microparticle may have a core with at least one active ingredient.

As used herein “unit dose” may refer to a physically discrete unit containing a predetermined quantity of an active material calculated to individually or collectively produce a desired effect. A single unit dose or a plurality of unit doses can be used to provide the desired effect, such as recovery effect and/or therapeutic effect.

As described herein, in some embodiments, the active agent of the core may be mononuclear, multi-nuclear or incorporated in a slow-release or controlled release or sustained release matrix. The core may also incorporate intestinal permeability enhancing agents.

In some embodiments, the plurality of microparticle comprises microparticles which have the same or different active agent, e.g., at least 1%, 5%, 10%, 20%, 30%, 40%, 50%, 60%, 70%, 80%, 90%, 95%, 98%, 99%, 99.9%, or all of the microparticles have the same active agent.

In some embodiments, the composition is the solid form.

In some embodiments, the composition is in the form of a powder. In some embodiments, the composition is in the form of a capsule. In some embodiments, the composition is in the form of a tablet. In some embodiments, the composition is in the form of a health/sports bar.

Non-Dry Composition Form:

In some embodiments, the disclosed composition has a non-dry form, i.e. comprises a liquid (e.g., 1% or more, by weight).

In some embodiments, the composition is in the semi-solid form.

The term “semi-solid” may refer to a material that is a mixture of liquid and solid phases, for example, such as particle suspension, colloidal suspension, emulsion, dispersion, gel, or micelle.

In some embodiments, the disclosed composition comprises e.g., at least 0.1%, 0.5%, 1%, 2%, 5%, 10%, 20%, 30%, 40%, 50%, 60%, 70%, 80%, 90%, 95%, or 99% by weight or by volume, of liquid, including any value therebetween.

In some embodiments, the microparticles in the liquid are in the range of from 0.01% to 99% (w/w or v/v). In some embodiments, the microparticles in the liquid are in the range of from 0.25% to 50% (w/w or v/v). In some embodiments, the microparticles in the liquid are in the range of from 0.50% to 33% (w/w or v/v). In some embodiments, the microparticles in the liquid are in the range of from 1% to 20% (w/w or v/v). In some embodiments, the microparticles in the liquid are 1% , 2%, 3%, 4%, 5%, 6%, 7%, 8%, 9%, 10%, 11%, 12%, 13%, 14%, 15%, 16%, 17%, 18%, 19% or 20% (w/w or v/v), including any value or range therebetween.

In some embodiments, the microparticles are dispersed in the liquid. In some embodiments, the liquid further comprises one or more supplementary ingredients. In some embodiments, the supplementary ingredients comprise one or more non-encapsulated active agents, flavor ingredients, or any combination thereof. Non-limiting exemplary supplementary ingredients include sweeteners such as aspartame, sodium saccharin, saccharin, stevia, glycyrrhizin, potassium acesulfame; acidulants such as citric acid, tartaric acid and malic acid; flavoring such as menthol, peppermint oil, peppermint, orange, lemon-lime, lemon and strawberry; and colorants such as caramel, annatto extract pigment, β-carotene and beet red.

In some embodiments, the composition is in the form of a gel.

The term “beverage” refers to a substantially aqueous drinkable composition suitable for human or other mammal consumption. In some embodiments, the beverage comprises at least 85% water by weight of the beverage, or at least 90% or from 95% to 99.9% water, by weight.

The term “gel”, as used herein is well accepted in the art, describes a semisolid composition for oral consumption. This term further encompasses compositions including a solid network that encages a liquid phase therewithin.

In some embodiments, the shell layer further comprises one or more flavor ingredients.

In some embodiments, one or more flavor ingredients and the non-encapsulated active agents are dispersed and/or dissolved in the liquid

As described hereinabove, in some embodiments, the disclosed encapsulated microparticles are inert or materially inert in terms of flavor and/or odor as a dispersed suspension in the liquid, (e.g., drink, beverage, gel, or foam) and pass through the mouth and esophagus without substantially releasing its contents. By “without substantially releasing” it is intended to mean releasing of e.g., no more than 33%, no more than 25%, no more than 20%, no more than 15%, no more than 10%, no more than 5% or no more than 1%, including any value in between.

In some embodiments, the liquid is water. In some embodiments, the liquid is alcohol. In some embodiments, the liquid comprises water and ethanol. In some embodiments, the liquid is any liquid approved for human consumption other than water or alcohol.

In some embodiments, the composition is in the form of a non-dry formulation.

In some embodiments, the composition is in the form of a beverage. In some embodiments, the composition is in the form of a shake. In some embodiments, the beverage is a gel. In some embodiments, the beverage is foam. In some embodiments, the beverage is a ready-to-consume drink. In some embodiments, in order to provide a reasonable shelf-life for the ready-to-consume drink version, more than a single encapsulation coating in the shell layer is required, as described hereinabove.

In some embodiments, the liquid is substantially devoid of liquid other than water. In some embodiments, the liquid is substantially devoid of a liquid other than water or alcohol.

In some embodiments, the liquid further comprises one or more ingredients selected from, but are not limited to, colorants, thickeners, flavoring agents, stabilizers and the like.

In some embodiments, the liquid comprising water and ethanol, comprises e.g., 1%, 10%, 20%, 30%, 40%, 50%, 60%, 70%, 80%, or 90% alcohol, including any value or range therebetween.

In some embodiments, the alcohol is ethanol. In some embodiments, the alcohol is methanol.

In some embodiments, the microparticles are dispersed in the liquid medium. In some embodiments, the microparticles are suspended in the liquid medium.

In some embodiments, when the encapsulated microparticles are reconstructed into a liquid-based or liquid-containing composition, the microparticles are suspended in the liquid and are homogenously distributed therein (e.g., not materially floating at the surface of the liquid and not materially settling at the bottom of the container).

Hence, in some embodiments, the disclosed microparticles are capable of maintaining their capacity to disperse and re-disperse in the liquid-containing medium.

In some embodiments, the terms “disperse”, “dispersive” or any grammatical derivative thereof, refer to the tendency of a particulate matter not to become or stay as agglomerates, clumps or chunks of mass, but rather become and/or regain a state of discrete, non-aggregated free particles.

In some embodiments, “dispersing” further refers to the act of crumbling, breaking apart or otherwise separating the microparticles which have formed clumps, soft agglomerates and soft aggregates back into the un-clumped particle.

In some embodiments, the term “suspension”, or any grammatical derivative thereof, as used herein, refers to a heterogeneous mixture of a solid in fine solute-like microparticles dispersed in a liquid or solvent-like phase. Typically, a suspension will have a tendency to settle, namely the fine particles of the solid matter may have the tendency to precipitate after a period of time. This period of time may depend on many factors, such as the substances of the microparticles and the liquid, the temperature and other physical parameters like stirring and shaking, and the presence of other substances, such as dispersing agents, emulsifiers, surface-active agents, thickeners and the likes.

In some embodiments, the term “suspension” as used herein, is similar to the term dispersion, with the proviso that the media is a condense medium, typically a liquid. Thus, a suspension may be a collection of discrete and separated particles dispersed in a liquid-based medium. The capacity to re-disperse is applicable also in a liquid-based media, namely the capacity to re-suspend, or go from a precipitant to a suspension reversibly and reproducibly.

Thus, according to some embodiments of the present invention, the composition is in the form of dispersion, or as suspension of the disclosed microparticles in liquid or liquid-containing medium.

In some embodiment, the liquid-based medium further comprises one or more surface-active agents, also referred to as “surfactant”.

In some embodiments, the term “surfactant”, as used herein, refers to an acceptable material which imparts emulsifiability, stability, spreading, wetting, dispersibility or other surface-modifying properties.

In some embodiments, the surfactant is selected from, but is not limited to, cationic surfactants, anionic surfactants, non-ionic surfactants, amphoteric surfactants, and zwitterionic surfactants.

Non-limiting exemplary surfactants are Sodium dodecyl sulfate (SDS) and Polyvinylpyrrolidone (PVP), fatty substances (e.g., lecithin), a cholic acid or a derivative thereof, and bioavailability enhancers (e.g., D-α-Tocopherol polyethylene glycol succinate, TPGS).

In some embodiments, the disclosed composition, in the form of dispersion or suspension (e.g., a beverage, a drink, a shake, a gel, a foam or any other non-dry delivery media) is characterized by a defined pH, where such defined pH prevents or materially prevents premature dissolution and release of the microencapsulated actives into the non-dry media.

The term “pH” refers to the quantitative measure of the acidity or alkalinity (basicity) of liquid solutions or dispersions.

In some embodiments, the pH of the non-dry composition is below 7. In some embodiments, the pH of the non-dry composition is below 5. In some embodiments, the pH of the non-dry composition is 2, 3, 4, 5, 6, or 7, including any value or range therebetween.

In some embodiments, the pH refers to the pH of the non-dry composition. In some embodiments, the pH refers to mouth pH. In some embodiments, the pH refers to esophagus pH. In some embodiments, the pH refers to stomach pH. In some embodiments, the pH refers to duodenum pH. In some embodiments, the pH refers to jejunum pH. In some embodiments, the pH refers to ilium pH. In some embodiments, the pH refers to colon pH.

In some embodiments the desired value of pH is set by an addition of an acid (e.g., HCl) or a base (e.g., NaOH), or a salt thereof, to the liquid, or to a powder comprising the microparticles.

Bioactivity of the Composition:

In some embodiments, once passed the esophagus, the composition, depending on its disintegration or dissolution environmental trigger, begins to release its active ingredients (in a selective and controlled manner) in the stomach and/or in a selected region of the small intestines and/or in the colon, depending on the properties of the core or matrix, the properties of the coating layer or layers, the ratio between the core or matrix and the coating layer or layers (w/w or v/v) and the (w/w or v/v) ratio between the shell material and active agents.

In some embodiments, the disclosed composition is formulated for a subject. In one embodiment, the term “subject” refers to any member of the mammal.

In some embodiments, the term “subject” is to be read to include “human”, “individual”, “consumer”, “animal”, “patient” or “mammal” where context permits.

In some embodiments, the term “subject” defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters, guinea pigs, and so on.

In some embodiments, a release of the active agent into the subject is a result of an environmental trigger and specific ingredients in the shell and/or the core.

In some embodiments, the term “environmental trigger” refers to a change in one or more environmental conditions (e.g., physiological condition(s)) sufficient to initiate degradation in the encapsulating materials of the shell and/or the core and/or the matrix, the change leading to a pre-defined release profile of the bioactive agent(s) encapsulated therein. In some embodiments, the environmental trigger is time, or in another embodiment temperature, or in another embodiment moisture content, or in another embodiment pressure, or in another embodiment pH, or in another embodiment ionic strength, or in another embodiment enzymatic activity, or in another embodiment mechanical conditions (e.g., grinding by GI motility), or in another embodiment any combination thereof.

In some embodiments, an ingredient or combination of ingredients in the shell dictates targeted release of the active agent e.g., in the stomach, duodenum, jejunum, ilium, secum or the colon and the controlled release profile during a specified period of time (e.g., 5 minutes to 8 hours).

In one embodiment the environmental condition change may be by a change of ±2.5% in the reference environmental condition, or in another embodiment a change of ±5% in the reference environmental condition, or in another embodiment a change of ±10% in the reference environmental condition, or in another embodiment a change of ±15% in the reference environmental condition, or in another embodiment a change of ±20% in the reference environmental condition, or in another embodiment a change of ±25% in the reference environmental condition, or in another embodiment a change of ±30% in the reference environmental condition, or in another embodiment a change of ±35% in the reference environmental condition, or in another embodiment a change of ±40% in the reference environmental condition, or in another embodiment a change of ±45% in the reference environmental condition, or in another embodiment a change of ±50% in the reference environmental condition, or in another embodiment by a change of more than ±50% in the reference environmental condition.

In one embodiment, the reference environmental condition is time, or in another embodiment temperature, or in another embodiment moisture content, or in another embodiment pressure, or in another embodiment pH, or in another embodiment ionic strength, or in another embodiment enzymatic activity, or in another embodiment any combination thereof.

In some embodiments, the shell surrounding the core or matrix incorporating an active compound (agent) is specifically designed to degrade, or in another embodiment, undergo controlled release, as a response to exposure to the change in environmental condition, which is in one embodiment time, or in another embodiment temperature, or in another embodiment moisture content, or in another embodiment pressure, or in another embodiment pH, or in another embodiment ionic strength, or in another embodiment enzymatic activity, or in another embodiment any combination thereof.

In some embodiments, the shell is a protecting layer which provides protection to the encapsulated active agent, so that the active agent shall maintain or materially maintain its bioactive properties in hostile pre-consumption or post-consumption conditions. In another embodiment, based on a triggering event for the initiation of core, matrix or shell dissolution or disintegration or swelling, the at least one core, matrix or outer protection layer is dissolved, disintegrated or swelled, or in another embodiment outer protection layers are dissolved, disintegrated or swelled and the “dormant” active agent will be released and become physiologically active.

In some embodiments, selective release can be affected using different types of matrix or coating layer (e.g., that are pH independent), and their disintegration is triggered in the presence of enzymes, and augmented mechanical GI tract forces.

For example, different enzymes may be present in the stomach, duodenum, jejunum, ilium and colon, each enabling a specific and different triggering event.

For example, and without limitation, core, matrix or a coating layer that are insoluble in non-enzymatic acidic conditions can be fabricated from coating compositions based on Prolamin proteins (Zein, Gluten, Kafirin, etc.), Shellac, fats (Coconut oil, Palm oil, etc.), and any combination thereof. Such core, matrix or a coating layer can be used to selectively release the active agent from the core.

In some embodiments, a first (and inner) encapsulation coating layer in the shell can control the release of the active agent as described above and at least one additional encapsulation layer can be used to further seal the core and the first encapsulation layer from the non-dry composition (e.g., in the gel or the ready-to-drink (RTD) product).

Such at least one additional outer encapsulation coating layer, or shell, may be designed for maximum sealing.

In some embodiments, the shell is breached under the enzymatic and/or mechanical grinding conditions of the stomach, the small intestines or the colon, thereby exposing the inner encapsulation layer and its selective dissolution profile thereof.

In some embodiments, the outer (second) encapsulation coating layer can include materials which are insoluble in acidic, non-enzymatic conditions, but are rapidly dissolved/disintegrated under the enzymatic and/or mechanical grinding conditions of the stomach, the small intestines or the colon. Such materials are described hereinthroughout, and include, but are not limited to, Prolamin proteins (Zein, Gluten, Kafirin, etc.) and fats (Coconut oil, Palm oil) and any combinations thereof.

In some embodiments, the coating/shell is dissolved in the presence of specific digestive enzymes, for example, and without limitation, glycoside hydrolase (e.g., chitinases and chitosanases), pepsin, trypsin, amylase, lipase and liver enzymes.

In some embodiments, a coating dissolved in the presence of specific digestive enzymes can be based on Prolamin proteins (e.g. Zein, Gluten, Kafirin), Gelatin, Soy proteins, Pea proteins (Globulin), Vegetable proteins, fats, Starches, Dextran, Maltodextrin, Cyclodextrin, Whey and Casein, and any combination thereof.

In some embodiments, a coating dissolved or disintegrated in the presence of a specific digestive enzyme of the small intestines include, but are not limited to: Guar gum, gum Arabic, Pectin and Amylose, and any combination thereof. Such coatings can be used to release the active agent of core of the microparticle in the small intestine.

In some embodiments, a coating dissolved or disintegrated in the presence of a specific enzyme of the colon include, but not limited to, Starches, Guar gum, Pectins, Chitosans, Alginates, Hydrogels, and any combination thereof. Such coatings can be used to release the active agent of the core of the microparticle in the colon.

In some embodiments, a coating dissolved or disintegrated under low pH conditions include, but are not limited to, Polymethacrylates. Such coatings can be used to release the active agent of core of the microparticle in the stomach.

Other coatings that facilitate slow-release/controlled release/sustained release, independent of enzymatic and pH conditions include, but are not limited to, Ethyl Cellulose and other cellulose derivatives. Such coatings can be used for controlled release of an active agent throughout the GI tract.

In some embodiments, the shell of the microparticles can be non-uniform e.g., to allow selective dissolution of the microparticles and prolonged selective release of the active ingredient. Several approaches for enabling selective release of active ingredients from encapsulated microparticles are described in the Examples section that follows.

In some embodiments, the active agent is designed in a way whereby the release of the active agent occurs before entering a gastrointestinal tract GI system of the subject (e.g., human or animal) consuming the formulation.

In some embodiments, the release may be while in contact with specific part(s) of the gastrointestinal tract.

In some embodiments, the term “protect”, or any grammatical derivative thereof, is meant to refer to the encapsulation of the shell protecting the active agent, at least partially, during its passage through the stomach, such that sufficient amounts of the active agent are still bioactive and are capable of promoting the required positive health promotion or the required benefits, whether e.g., acute, prophylactic or chronic.

In some embodiments, the term “protect” and any grammatical derivative thereof, further refers to the encapsulation of the shell protecting the active agent, thereby eliminating or significantly minimizing unpleasant odor and/or taste of the active agent.

As mentioned hereinabove, in some embodiments, the shell surrounding an active agent is specifically designed to degrade or disintegrate or swell, or in another embodiment, undergoes controlled release, as a response to exposure to the change in environmental condition or a combination of environmental conditions.

In some embodiments, the term “controlled release” means control of the rate of dissolution of the active agent in a body fluid (e.g., in the gastrointestinal tract) such that it is slower than the intrinsic dissolution rate of the active agent (e.g., pharmaceutical API, an active agent or a dietary supplement) in such a medium. It may otherwise or additionally mean a delayed release of the active agent. This effect results in the active agent being released into the solution over a longer period than would be achieved if the active agent were administered without control of its release pattern and/or after an initial delay.

In some embodiments, the pattern of controlled release achieved by the present composition is known as sigmoidal.

In some embodiments, “sigmoidal pattern” refers to a release profile exhibiting (a) an optional lag time from administration during which no specific active agent or very little amount of a specific active agent (e.g., less than 33%, 25%, 20%, 15%, 10%, 5%, 2% or 1%, by weight) is released, followed by (b) a phase where the rate of the active agent release increases, followed by (c) a phase where the rate of the specific active agent release decreases towards zero as the amount of the active agent in the composition is exhausted or materially exhausted.

In some embodiments, the changeover from phase (b) to phase (c) occurs when at least 50% by weight of the active agent has been released.

It is to be understood that the disclosed composition may incorporate e.g., one two several dozens of active agents, each specific active agent may be present in the composition in one or more core, matrix & core-shell and/or shell encapsulation combination, each combination having a different dissolution trigger or different dissolutions triggers and featuring a different release profile.

The disclosed composition may include a particle with multiple active agents which share an identical core, matrix, core-shell and/or shell providing a specific trigger event and a specific release profile.

The disclosed composition may include particles, where active agents, based on its water solubility (e.g., all water-soluble active agents, all partially water soluble active agents and all water insoluble actives) are grouped into several particles, each particle type having its own trigger and release profile.

In some embodiments, the composition comprises at least two (e.g., 3, 4, 5, 6, 7, 8, 9,10, 11, 12, 13, 14, 15, 16, 17, 18, 19, 20, 21, 22, 23, 24, 25, 26, 27, 28, 29 or 30) different microparticles in a plurality of the microparticle, wherein each microparticle comprises an identical, similar or different active agent being characterized by a different sigmoidal pattern of controlled release or sustained release of the active agent in a GI tract of a subject.

In some embodiments, at least one microparticle comprises at least two (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 38, 40, 42, 44, 46, 48 or 50, including any value therebetween) active agents, wherein each active agent is characterized by a different sigmoidal pattern of controlled release or sustained release thereof in a GI tract of a subject.

In some embodiments, there is provided a use of the disclosed composition for providing an effective dose of the at least one active agent to one or more target sites of GI tract of a subject. In some embodiments, the use is for oral administration.

In some embodiments, there is provided a kit, comprising the disclosed composition. In some embodiments, the kit is for oral administration. In some embodiments, the kit further comprises instructions for preparation and use. In some embodiments, the composition in the kit is in the solid form. In some embodiments, the kit further comprises at least one diluting solution.

In some embodiments, the components of the kit are maintained and/or stored in the form of solutions. In some embodiments, the composition may be maintained in a dry or solid form and the composition requires reconstruction and/or dissolution under particular condition(s), e.g., gentle blending or vigorous shaking.

By “storage” it is meant to refer to appropriate condition(s) for storing each one of the components of the kit in a single container, or dual chamber container (e.g. the composition and reconstituting liquid are isolated one from the other until reconstruction and consumption), or in separate containers. Container may be any container made of inert materials that do not interact with the content of each component and that are stable under the storage conditions. Thus, suitable containers for storing each components of the kit include a sachet, a bag, a can, a bottle, a vial, a large volume parenteral, a small volume parenteral, or a cassette. Storage conditions may include sterile or aseptic conditions, i.e. each component is preferably maintained sterile or aseptic. Alternatively, the content of each container may be readily sterilized.

In some embodiments, there is provided a method for treating a disorder, comprising administrating to an individual the disclosed composition.

In some embodiments, the method of treating a health or medical condition associated with any temporary, transient, acute or chronic disease, medical condition, disorder, or nutritional deficiency in a subject in need thereof comprises administering to the subject a therapeutically or nutritionally effective amount of the disclosed composition.

In some embodiments, the subject is a human. In another embodiment, the subject is a human suffering from a temporary, transient or chronic disease, disorder, health condition, or a nutritional deficiency.

In some embodiments, the composition is specifically formulated for controlled release of the at least one active agent in a gastrointestinal (GI) tract of a subject.

The Process:

Several types of micro-encapsulation approaches can be used to encapsulate the active agent (e.g., pharmaceutical API, natural active or dietary supplements) of the disclosed composition. Non-limiting examples of such approaches include encapsulating active agent(s) (e.g., pharmaceutical API, active ingredient, natural active or dietary supplement raw materials which are frequently characterized by a foul taste odor; or low water solubility) with enteric and/or other encapsulating materials in a fluidized bed; spray-drying a mixture of a dietary supplement and enteric/encapsulant ingredients; and/or; freeze-drying a mixture spray-drying a mixture of a dietary supplement and enteric/encapsulant ingredients, and then grinding it to the desired final particle size/diameter.

In some embodiments, a multi-layer encapsulation can be achieved. In some embodiments, a multi-layer encapsulation can be achieved in a single step e.g., using a 2-fluid, 3-fluid, 4-fluid or 5-fluid nozzle in a spray dryer; or a combination of spray drying and fluid bed coating

In some embodiments, the two-step process allows to generate the core in either a spray dryer or by grinding the active agent or a matrix incorporating the active agent to a pre-desired size (e.g., diameter), and then to use a fluidized bed to generate the at least one coating layer. In some embodiments, the core or matrix may be generated in any of a variety of methods known in the art, and then a single additional step of multiple steps in a fluidized bed of similar equipment (like a mixer with injected solution) is used to create the at least one shell/encapsulating layer.

The Examples section below describes several approaches for encapsulating the pharmaceutical API, active ingredient, natural active or dietary supplements of the present invention and composition. The advantages of the technological approach is that given optimal parameters, it enables the generation of an effective isolative coating(s) of active agent(s) which are foul tasting and/or water soluble, even in cases of very small (i.e. microsized) particles. The effectively isolative coating may be resistant or materially resistant to acidic non-enzymatic environmental conditions, may it be in a dry composition or a non-dry composition.

According to an aspect of some embodiments of the present invention there is provided a process of preparing any of the compositions described herein.

In some embodiments, the process comprises:

mixing the active agent and at least one from the ingredients (i) (ii) and (iii) as defined hereinabove;

dissolving the one or more enzymatically-degradable water-insoluble ingredients in a solvent (e.g., alcoholic or other organic solvent) either purely, or in combination with water, thereby forming a shell solution;

concurrently spray-drying the core solution and the shell(s) solution, each in its own separate channel, thereby forming the microparticle.

In some embodiments, the mixing of the active agent and the at least one encapsulating ingredients (i.e. water-insoluble, enzymatically-degradable coating, liquid-swellable coating, pH-dependent coating, enteric coating or any combination thereto) as defined hereinabove, is performed in a liquid or non-dry solution, thereby forming a core solution.

Herein, “liquid” may refer to a solvent or a mixture of solvents. In some embodiments, the solvent is or comprise one or more organic (pharma grade or food grade) solvents and/or water.

In some embodiments, the pharma grade or food grade solvent is selected from, but is not limited to, methanol, ethanol, acetone and ethyl acetate, or other appropriate solvents e.g., approved by the regulatory agency (e.g., FDA) for pharmaceutical and/or food grade compositions, or their mixtures or combination thereof.

In exemplary embodiments, for preparing, the ratio (w/w or v/v) of the active agent (s) and the shell is 20:1, 15:1, 10:1, 9:1, 8:1, 75: 6:1, 5:1, 4:1, 3:1, 2:1, 1:1, 1:2, 1:3, 1:4, 1:5, 1:6, 1:7, 1:8, 1:9, 1:10, 1:15, 1:20, 1:30, 1:40 or 1:50, including any value therebetween.

Herein, the ratio may relate to the percentage of the raw material incorporating the active(s), or in some embodiments, to the percentage of the active agent per se.

In exemplary embodiments, in a particle having a size of 50 to 60 microns, the active agent (s) are in the range 5%-66% and the shell is in the range of 10%-30%, by weight of the total particle.

In additional exemplary embodiments, in the 10 microns particle (manufactured by e.g., Buchi B290), the active agent(s) or the raw material incorporating the active(s) are in the range of 1%-45% and the shell is about 10%-50%, by total weight of the particle.

In additional exemplary embodiments, in 90-100 micro-sized particles, the active agent(s), or the raw material incorporating the active(s), are in the range of 5% to 60% and the shell is in the range of 10%-25%, by total weight of the particle.

In exemplary embodiments, the total of water-insoluble enzymatically-degradable coating, water-sellable coating, pH-dependent coating and enteric coating ingredients, and the total of active agent (s) are in ratio of about 3:1:0.5, by weight. Herein “about” may refer to with ±10%, ±20%, or ±30% or ±40% variation of any ratio thereof.

In some embodiments, the one or more enzymatically-degradable water-insoluble ingredients are in amount that ranges from 1% to 35%, by total dry weight of the core solution. In some embodiments, the one or more enzymatically-degradable water-insoluble ingredients are in amount that ranges from 1% to 15%, by total dry weight of the core solution. In some embodiments, the one or more enzymatically-degradable water-insoluble ingredients are in amount that ranges from 8% to 12%, by total dry weight of the core solution.

In some embodiments, the shell solution comprises water-insoluble ingredients such as slow release polymers. In some embodiments, the shell solution further comprises a pH dependent material, for example, and without limitation, phthalate, cellulose, and any derivative thereof.

In some embodiments, the shell solution comprises enteric coating ingredient(s) (e.g., in an amount that ranges from 1% to 33% solids) which are dissolved in an organic solvent solution or water or any combination thereto. Herein “water” may include water-based emulsions.

In exemplary embodiments, the shell solution and the core solution are sprayed, each in a separate fluid channel in a closed-loop or in an open loop (for example, by a Buchi B-290 equipped with a 3-fluid nozzle: a gas channel: nitrogen or air, a fluid channel for the core solution and a fluid channel for the shell solution), optionally without the aid of a humidifier.

In some embodiments, for manufacturing the product in standard open-loop (rather than a closed-loop spray dryer requiring a nitrogen atmosphere for the prevention of explosion) systems (e.g., spray drying and/or fluidized bed), the combination of the core composition and the shell composition, incorporating aggregate alcohol content under 25% and preferably under 20%, may be used.

Hence, in additional exemplary embodiments, the core comprises a small amount of an organic solvent (such as, but not limited to alcohol) with or without water. In additional exemplary embodiments, the shell comprises a small amount of organic solvent (alcohol), with or without water.

By “small amount”, it is meant to refer to an amount that ranges from 0% to 60%, by weight, for example, 0%, 5%, 10%, 15%, 20%, 25%, 30%, 35%, 40%, 45%, 50%, 55%, or 60%, including any value and range therebetween.

In exemplary embodiments, “small amount” refers to at most 25%.

In some embodiments, the shell solution and/or the core solution comprise at least 95% of organic solvent(s).

The organic solvent(s) may necessitate, in some embodiments, the use of a closed-loop spray dryer (e.g., a spray dryer with nitrogen atmosphere and an integral humidifier unit for the cooling and collection of sprayed alcohol) and/or a fluid bed.

In some embodiments, the process comprises:
(A) blending the active agent and at least one from (i) and (ii) (as defined above under “The composition”) in a solution comprising organic solvent (e.g., alcohol) and/or water, thereby forming a core solution;
(B) dissolving the one or more enzymatically-degradable water-insoluble ingredients in an alcoholic solvent and/or water or any combination thereto, thereby forming a shell solution;
(C) Spray drying the core solution to make the core particle; and
(D) Encapsulating the core particle with the shell solution, using a fluidized bed.

Further embodiments of the process are described in the Examples section that follows.

General:

As used herein, and unless stated otherwise, the term “about” refers to ±25%.

The terms “comprises”, “comprising”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical aesthetical, or nutritional symptoms of a condition or substantially preventing the appearance of clinical, nutritional or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

The Examples (and the corresponding Figures) below describe and analyze the continuous dissolution testing of the sample (microparticles), in the following sequence, where the conditions of the sample are set and modified according to the conditions of the stage (referred to as: “station”):
(a) First station: The acidic (pH 2.5) non-enzymatic conditions of the beverage, for 30 minutes.
(b) The acidic and enzymatic conditions of the stomach, for 20 minutes.
(c) The conditions of the small intestines, for minutes 20.
(d) The conditions of the colon, for minutes 20
Total: 90 minutes.

Reference is now made to the following examples, which together with the above descriptions, illustrate the invention in a non-limiting fashion.

### Example 1

### Preparation of the Microparticles

In exemplary procedures, the disclosed microparticles were prepared.

First variation: In exemplary procedures, a core solution was prepared. In exemplary procedures, the core (or matrix) solution contained at least two main components of an enzymatic-degradable coating, active agent and optionally contained enteric coating components in a ratio of 3:1:0.5, respectively.

The solution was an alcohol solution or a water based emulsion.

In exemplary procedures, a coating (shell) solution was prepared.

In exemplary procedures, the coating (shell) solution comprised a slow release polymer, optionally in combination with other ingredients that are pH dependent like phthalates.

In exemplary procedures, the dissolved enteric coating was in an amount of 10-20% solids in the coating (shell) solution.

In exemplary procedures, the core and shell solutions were sprayed in a Buchi B-290 equipped with a 3-fluid nozzle in a closed-loop (e.g. with Nitrogen atmosphere) or an open loop depending on the specific formulations and the amount of organic solvents (e.g., alcohols) in the solution.

In exemplary procedures, the ratio in the formulation was: active agent: core matrix: shell coating 1:3:1.5-5, respectively.

Second variation (also referred to as: “reverse coated particle”):

In this variation, the shell solution contained a water based emulsion of a slow release polymer and an enteric coating ingredient. In this variation, the core solution was also a water based emulsion comprising the active material.

In exemplary procedures, core and shell formulations that aggregately contained maximum 25% organic solvent (alcohol) were produced, allowing the use of an open-loop spray (e.g. with ambient air atmosphere) drying system with no humidifier. In this method, the alcohol and water were evaporated (this open spray drying system is more common in the food industry, and is also broadly used in the pharmaceutical industry).

In this case, the core and the shell solutions combined contained low amount of alcohol, minimum as possible from 0% to 25% combined

In exemplary procedures, the spraying rate was set.

In exemplary procedures, the coating solution contained the enzymatic-degradable coating components in a water and alcohol solution of max 60% alcohol (depends on the solubility of the coating ingredients).

Typically, the total % of alcohol in both solutions did not exceed 25%.

In exemplary procedures, the spray ratio of the two solutions was fixed to obtain a max 25% alcohol in both sprayed solutions. Increasing of the particle size enables to reduce the amount of the coating and by that reducing the aggregate alcohol levels in the combination of core and shell formulation being sprayed concurrently.

Specific Compositions:

Two feed solutions were prepared:

A core solution containing 3 g of Zein—a water-insoluble, enzymatically-degradable Prolamin protein, 1 g of vitamin B6 and 1 g 0.5 g of Shellac dissolved in 100 g of 70% of alcohol/30% water solution, and a coating solution containing 10 g of Ethyl cellulose dissolved in 100 g Ethanol.

The total solids content was 4.5% for the core solution and 10% solids for the coating solution.

The core matrix solution and the coating solution were concurrently spray dried in a 2:1 ratio using a 3-fluid nozzle fitted to a Buchi-290 Mini spray drier (Flawil, Switzerland) operating in a close loop. The inlet temperature was 130 degrees C. and the aspirator was set to100%. The outlet temperature was 95-100 degrees C.

The dissolution test of the powder obtained from the one-step spray drying process was performed with a dissolution tester Varian k7000.

Table 1 summarize exemplary microparticle.

TABLE 1

| | | 10 microns | | 50 microns | | 100 microns | |
|---|---|---|---|---|---|---|---|
| | | Low | High | Low | High | Low | High |
| CORE | ACTIVE | 1.00% | 25.00% | 1.00% | 40.00% | 1.00% | 70.00% |
| | + | | | | | | |
| | ENTERIC | 1.00% | 40.00% | 1.00% | 50.00% | 1.00% | 50.00% |
| | Shellac, chitozan, phtalates (Eudragits) | | | | | | |
| | combinations with Zein protein | | | | | | |
| | + | | | | | | |
| | ENZYMATIC | | | | | | |
| ENCAPSULATION | celluilose derivative, Eudragit, phtalates | 8.00% | 80.00% | 8.00% | 50.00% | 8.00% | 25.00% |

“High” and “Low” refer to percent by weight.

The average dissolution release of the active agents in various media are as follows: beverage: max 10% in 0-30 min. acidic: max 20% in 20-50 min. enteric:

80-100% in 50-90 min.

### Example 2

### Characterization of the Microparticles Release Profile in a Sequence Comprising: (1) Acidic & Non-Enzymatic Condtions (2) Stomach Conditions (3) Intestinal Conditions

The tests were performed according to a standardized static in vitro digestion method suitable for food—an international consensus (Food Function, 2014, 5, 1113-1124).

In exemplary procedures, the test fluid was 600 ml solution of water in pH 2.5 obtained by using HCl solution. The acidic water solution did not incorporate any enzymes.

The temperature in the well of the dissolution tester Varian k7000 was adjusted to 37° C., and the rotation speed of the paddle was at the first 30 seconds 250 rpm to simulate addition of powder to water to generate a beverage. After 30 minutes a gastric enzyme (such as pepsin 2000 u/ml activity) was added to the same well. After 20 minutes in the gastric solution the pH was changed and adjusted to 5.0 by using NAOH solution. After 20 minutes the pH 5.0 was changed and adjusted again to pH 7.0.

Sodium dodecyl sulfate (SDS) was added as well in cases of low solubility of the active agents. Table 2 summarizes the parameters of various exemplary procedures.

TABLE 2

| Time (min) | | Blending | | Pepsin |
|---|---|---|---|---|
| sampling | pH | (rpm) | SDS | enzyme |
| 15 | 2.5 | *— | − | − |
| 30 | 2.5 | — | − | − |
| 40 | 2.5 | 250 | − | + |
| 50 | 2.5 | 250 | − | + |
| 70 | 5.0 | 250 | + | + |
| 90 | 7.0 | 250 | + | + |

*In the first 30 seconds the composition in liquid was stirred in 250 rpm. Thereafter the stirring was stopped and turned on sporadically to simulate the intestinal regions.

Gastric Target Release

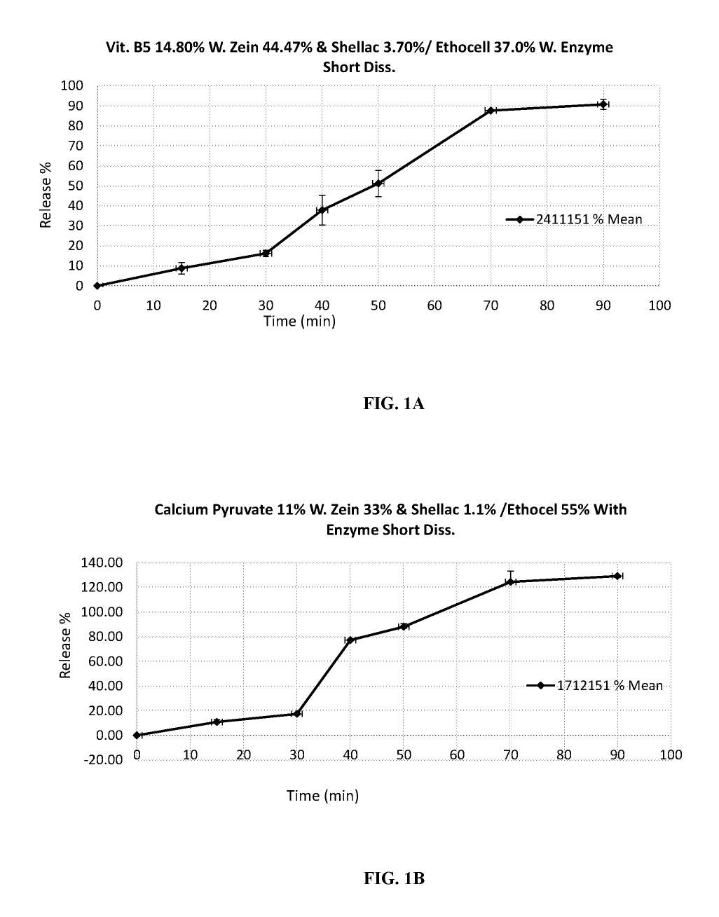

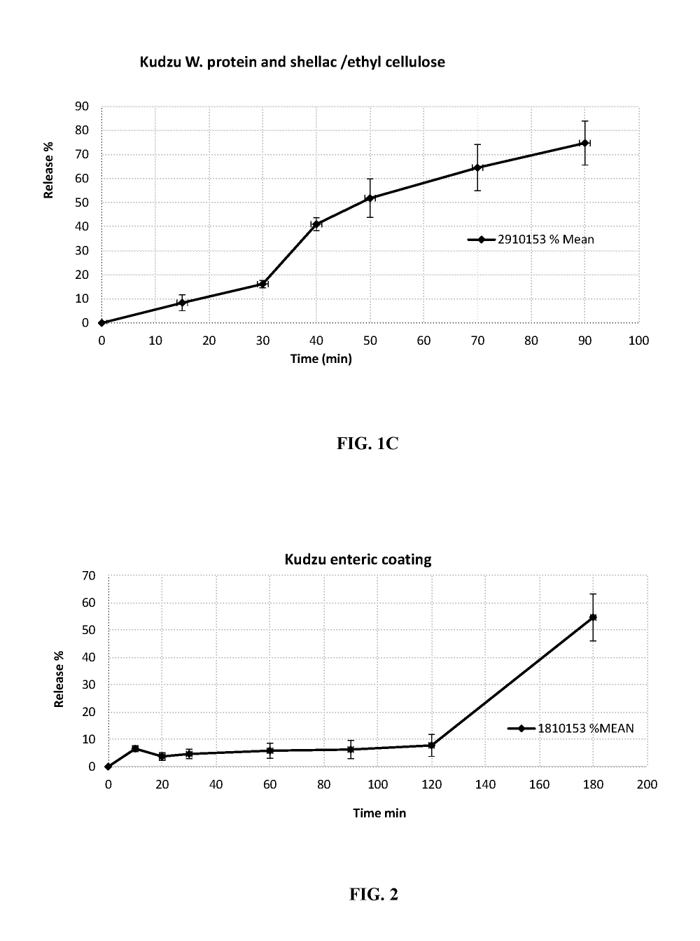

Examples of typical dissolution graphs of gastric target release are shown in FIGS. 1A-1C, showing the release profile of:

Vitamin B5 (FIG. 1A; the particle content, by weight: Vitamin B5 14.80%. Zein 44.47% & Shellac 3.70%/Ethocel 37.0%. Enzyme Short time Dissolution; pH over time: 0-30 min-beverage pH=2.5; 30-50 min-Gastric fluid pH=2.5; 50-70 min-Intestine pH=5.0; 70-90 min intestine pH=7.0);

Calcium pyruvate (FIG. 1B; the particle content, by weight: Calcium Pyruvate 11%. Zein 33% & Shellac 1.1%/Ethocel 55%)(pH over time: 0-30 min-beverage pH=2.5; 30-50 min-Gastric fluid pH=2.5; 50-70 min-Duodenum pH=5.0; 70-90 min intestine pH=7.0);

and of kudzu (FIG. 1C; the particle content: Kudzu (20% actives) and shellac 1%/ethyl ce11u1ose79%; pH over time as in FIG. 1B).

Examples of typical release profile of Kudzu of microparticles having enteric coating are shown in FIG. 2 (pH as in FIG. 1B).

Release Profile with Only One of the Excipients:

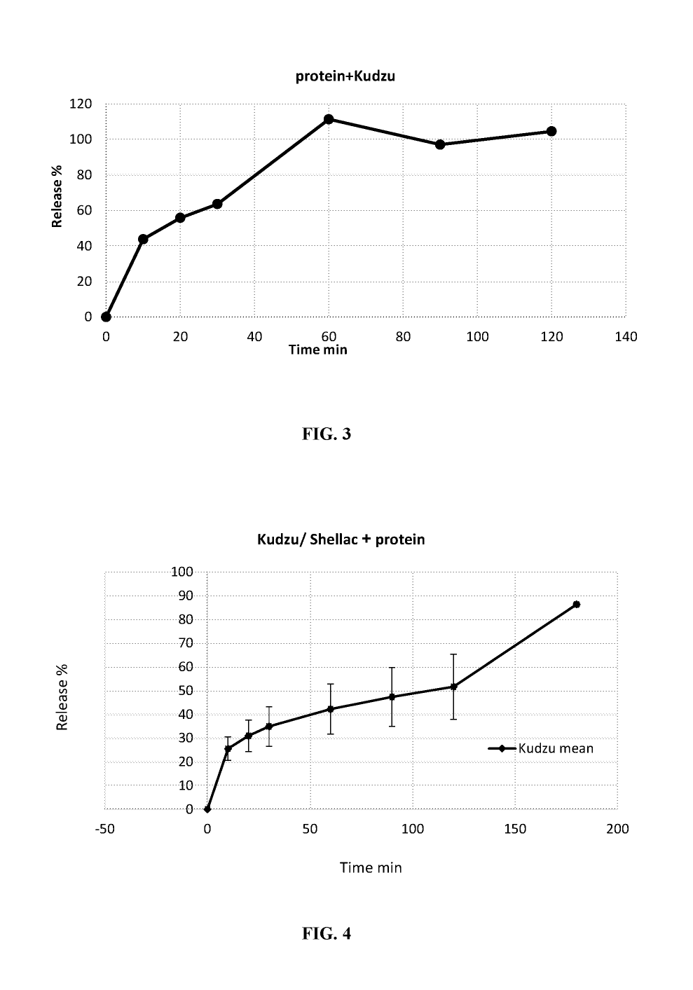

When trying to obtain the desired release profile with only one of the encapsulating materials, the desired release profile could not be achieved. For example, when only the Zein protein was used as an encapsulation material, an immediate release of the active agent (e.g., Kudzu) was probed, as shown in FIG. 3, with 60% of Kudzu being released after 30 minutes in the media simulating the beverage, whereas none or only minimum release was desired in the media simulating the beverage.

When generating a particle having an active agent and with only the core (Kudzu+Shellac 1%+Zein 4%) with no shell layer at all, the release profile could not be anticipated in advance, and the actual release profile of the Kudzu was too fast with more than 30% being released into the media simulating the beverage in the first 30 minutes, as shown in FIG. 4.

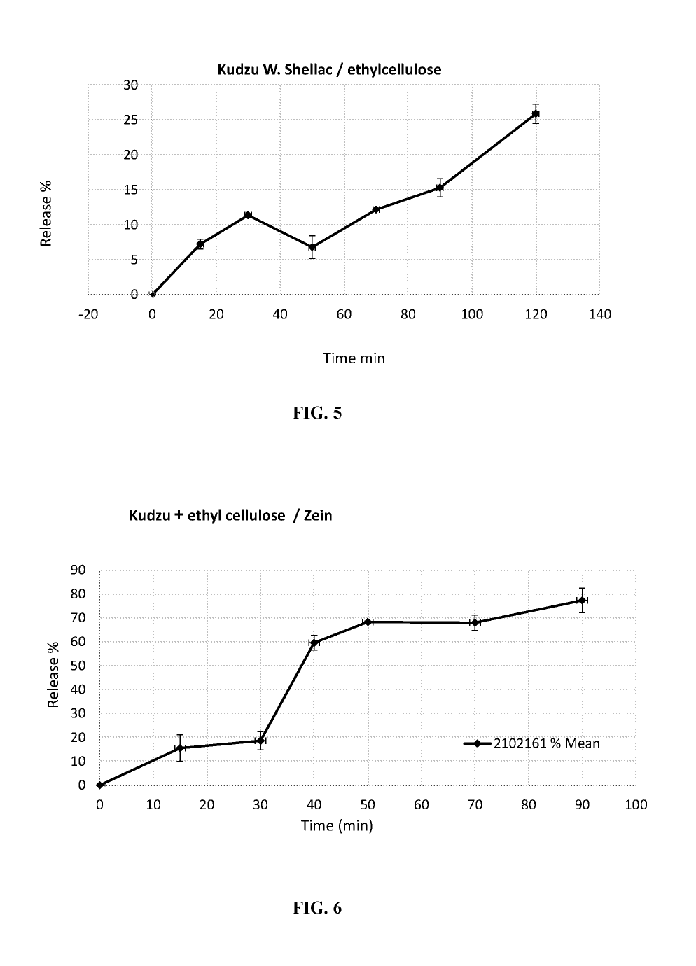

When using a core incorporating the enteric component without the water-insoluble, enzymatic-degradable component (Kudzu+Shellac 1%+Ethocel 10%), the desired release profile was not obtained, and almost no release of Kudzu during the desired dissolution time (45-60 minutes starting from the stomach and continuing in the small intestines) could be observed, as shown in FIG. 5.

Formulation in an Open System Spray Dryer with Reverse Coating:

FIG. 6 presents the release profile of the active agent (Kudzu) when the formulation was produced by in an open-loop system spray dryer with a reverse coating approach. The particle comprises Kudzu, ethyl cellulose and Zein.

### Example 3

### Exemplary Microparticles

In exemplary procedures, microparticles containing various amounts of active agents were prepared as shown in Table 3. In additional exemplary procedures, the microparticles were further mixed with additional non-encapsulated active agents, as further summarized in one specific example in Table 3.

TABLE 3

| | | Ingredients |
|---|---|---|
| 1 | EN* | N-Acetyl-L-cysteine |
| 2 | | Puerarin (Kudzu) |
| 3 | | Huperzia |
| 4 | | Guarana (Caffeine) |
| 5 | | Alpha lipoic acid |
| 6 | | Curcumin |
| 7 | | Piperine |
| 8 | | Quercetin |
| 9 | | Resveratrol |
| 10 | | Ginseng |
| 11 | | Gingerols (Ginger) |
| 12 | | White willow bark |
| 13 | | Grape seed |
| 14 | | Vitamin B1 |
| 15 | | Vitamin B2 |
| 16 | | Vitamin B3 |
| 17 | | Vitamin B5 |
| 18 | | Vitamin B6 |
| 19 | | Vitamin B9 |
| 20 | | Vitamin B12 |
| 21 | | Vitamin C |
| 22 | | Magnesium citrate |
| 23 | | Zinc citrate |
| 24 | | L-Carnitine |
| 25 | | Sodium pyruvate |
| 26 | Non- | Vitamin B1 |
| 27 | Encapsulated | Vitamin B2 |
| 28 | | Vitamin B3 |
| 29 | | Vitamin B5 |
| 30 | | Vitamin B6 |
| 31 | | Vitamin B9 |
| 32 | | Vitamin B12 |
| 33 | | Vitamin C |
| 34 | | Magnesium citrate |
| 35 | | Zinc citrate |
| 36 | | L-Carnitine |
| 37 | | Sodium pyruvate |
| 38 | | Fructose |
| 39 | | Glucose |
| 40 | | Betaine |
| 41 | | Choline |
| 42 | | N-Acetyl-L-cysteine |
| 43 | | Puerarin (Kudzu) |
| 44 | | Huperzia |
| 45 | | Guarana (Caffeine) |
| 46 | | Lemon powder |
| 47 | | Citric acid |
| 48 | | Sucralose |
| 49 | | Caramel |
| 50 | | Thickener (7HOF) |
| 51 | | Gum arabic |
| 52 | | Silicon dioxide |

*EN—Encapsulated

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A composition comprising at least one microparticle, wherein said at least one microparticle is in the form of a core-shell, and wherein said core comprises:
(a) at least one active agent; and
(b) at least one pH-triggered ingredient, at least one enteric ingredient, or at least one enzymatically-degradable ingredient, or any combination thereof; and
said shell comprises one or more water-insoluble and enzymatically-degradable ingredients, wherein the weight ratio of said active agent and the sum of (b) ranges from 1:10 to 10:1.

2. The composition of claim 1, wherein said shell further comprises at least one pH-triggered ingredient or at least one enteric ingredient.

3.-4. (canceled)

5. The composition of claim 1, wherein said active agent is selected from the group consisting of: pharmaceutical APIs, active ingredients, nutraceuticals, food supplements, food additives, herbals, plant extracts, medicaments, homeopathic agents, and any combination thereof.

6. The composition of claim 1, wherein said shell comprises two or more layers, wherein each layer comprises a different material.

7. The composition of claim 1, comprising said least one pH-triggered ingredient or an enteric ingredient, and said at least one enzymatically-degradable ingredient.

8. (canceled)

9. The composition of claim 1, comprising a plurality of microparticles.

10. The composition of claim 1, further comprising a liquid.

11. The composition of claim 10, wherein said liquid is selected from the group consisting of: water, organic solvent, alcohol, and any combination thereof.

12.-13. (canceled)

14. The composition of claim 1, further comprising one or more non-encapsulated active agents.

15. The composition of claim 1, wherein the active agent is selected from the group consisting of sugar, fructose, glucose, betaine, choline, cysteine, n-acetyl-1-cysteine, carnitine, kudzu, hovenia dulcis, dihydromyricetin, puerarin, huperzia, guarana, theophylline, alpha lipoic acid, curcumin, piperin, quercetin, resveratrol, ginkgo biloba, ginseng, bacopa monnieri, ginger, feverfew, butterbur, salicin, salicylic acid, Cannabidiol (CBD), vitamin B complex, vitamin C, vitamin D, vitamin E, magnesium salt, zinc salt, caffeine, theophylline, proanthocyanidin, or any combination thereof.

16. (canceled)

17. The composition of claim 10, having a pH value of below 7.

18. (canceled)

19. The composition of claim 1, wherein said one or more enteric ingredients comprise: a polymeric material selected from the group consisting of: hydroxypropylmethyl cellulose phthalate, hydroxypropylmethyl cellulose acetate succinate, methacrylic acid-methyl methacrylate copolymer, ethyl methacrylate-methyl methacrylate-chloro-trimethylammonium ethyl methacrylate copolymer, cellulose acetate phthalate, cellulose propionate phthalate, cellulose acetate maleate, polyvinyl acetate phthalate, polyvinyl alcohol phthalate, styrene-acrylic acid copolymer, methyl acrylate-methacrylic acid copolymer, or a water-soluble ingredient consisting of acetyl cellulose, methyl cellulose, hydroxypropyl cellulose, hydroxypropylmethyl cellulose, carboxymethyl cellulose, Carbomers, PEGs, Prolamin proteins (e.g., Zein, Gluten, Kafirin), Shellacs, fats (e.g., Coconut oil, Palm oil, Carnauba wax, Stearic acid, Sunflower oil), Gelatin, Soy proteins, Pea proteins (Globulin), Vegetable proteins, Starches, Dextran, Maltodextrin, Cyclodextrin, Whey, Casein, Guar gum, gum Arabic, Pectin, Amylose, Chitosans, Alginates, Hydrogels, Carbomers, Polymethacrylate, Ethyl Cellulose, Methyl Cellulose, or any combination thereof.

20. The composition of claim 1, wherein said shell comprises one or more materials selected from: Carbomers, PEGs, Prolamin proteins (e.g., Zein, Gluten, Kafirin), Shellacs, fats (e.g., Coconut oil, Palm oil, Carnauba wax, Stearic acid, Sunflower oil), Gelatin, Soy proteins, Pea proteins (Globulin), Vegetable proteins, Starches, Dextran, Maltodextrin, Cyclodextrin, Whey, Casein, Guar gum, gum Arabic, Pectin, Amylose, Chitosans, Alginates, Hydrogels, Carbomers, Polymethacrylate, Ethyl Cellulose and Methyl Cellulose.

21. The composition of claim 1, wherein at least one dimension of said microparticle is characterized by a diameter of 1 to 300 microns.

22.-24. (canceled)

25. The composition of claim 1, being in the form of: powder, a drink, a beverage, a shake, a foam, a capsule, a tablet, a bar or a gel.

26. The composition of claim 1, being characterized by a sigmoidal pattern of controlled release of at least one active agent within a gastrointestinal (GI) tract.

27.-28. (canceled)

29. A kit for oral administration, comprising:
(a) a composition-of-matter according to claim 1, and
(b) instructions for use.

30. A method for providing an effective dose of at least one active agent to one or more target sites within the gastrointestinal (GI) tract of a subject, comprising administering the composition of claim 1 to said subject, thereby providing at least one active agent to one or more target sites within the GI tract , optionally wherein said composition is in an oral dosage form.

31.-33. (canceled)

34. A process for preparing a microparticle in the form of a core-shell comprising:
(a) a core comprising at least one active agent and at least one from (i) and (ii):
(i) one or more pH-triggered or enteric ingredients, and
(ii) one or more enzymatically-degradable or water-insoluble ingredients, and
(b) a shell comprising one or more ingredients being characterized as enzymatically-degradable, and as water-insoluble ingredients, wherein:
a weight ratio of said active agents and a total of (i) and (ii) ranges from 1:1,000 to 10:1, the process comprising:
(A) blending said active agent and at least one from (i) and (ii) in a solution comprising organic solvent and/or water, thereby forming a core solution;
(B) dissolving said one or more enzymatically-degradable water-insoluble ingredients in an alcoholic solvent and/or water, thereby forming a shell solution; and
(C) concurrently spray-drying said solutions, each in a separate channel, thereby forming said microparticle;
optionally wherein any one of:
(i) said one or more enzymatically-degradable and/or water-insoluble ingredients are in an amount that ranges from 5% to 50%, by total weight of said a core solution.

35. The process of claim 34, wherein any one of:
(i) said one or more enzymatically-degradable and/or water-insoluble ingredients are in an amount that ranges from 5% to 50%, by total weight of said a core solution;
(ii) said organic solvent is selected from the group consisting of pharma-grade solvents and food-grade solvents;
(iii) said solvent is selected from ethanol, methanol, acetone, and any combination thereof:
(iv) any combination thereof.

36.-37. (canceled)
